(19) United States
(12) Patent Application Publication
(10) Pub. No.: US 2003/0100913 A1
(43) Pub. Date: May 29, 2003

(54) Depth adjustable cap of lancing device

(76) Inventor: Guoping Shi, Wuxi City (CN)

Correspondence Address: Jack E. Dominik, Dominik, Knechtel, Demeur & Samlan, Suite 225, 6175 NW 153rd Street, Miami Lakes, FL, 33014, US

(21) Appl. No.: 10/264,417

(22) Filed: Oct. 4, 2002

(30) Foreign Application Priority Data
Oct. 9, 2001 (CN) APPL. # 499080
Jun. 26, 2002 (CN) P# ZL 012 63172.8

(51) Int. Cl. A61B 17/14

(52) U.S. Cl. 606/181

(57) ABSTRACT

An improved depth adjustable cap of lancing device, including a lancet cover 3 with hole A 12 and an adjustable cover with hole B 13. The cover is set on the top of lancet cover 3. The two parts are joint up by screw structure and rotary position structure. Its character is: the adjustable cover is separated into a front cover 11 with hole B 13, a back ring 10 and two pieces of semi-circles. The semicircles from a set shape. The front cover 11 locates in one end of the shape and the back ring 10 located in the other end. There is a screw structure between one semi-circle and lancet cover 3. When rotating the adjustable cover, the rotary position structure makes the adjustable cover move intermittently. And “clicks” can be heard. The screw structure makes the hole B move along the axes to control the exposed lancet length. This program separates the original inner set into four simple parts. Then assemble them into a whole part and weld them with ultrasonic. Its characters: low down the manufacturing difficulty. Make it convenient to assemble. Product's quality is improved. Fact shows its effect is very obvious.

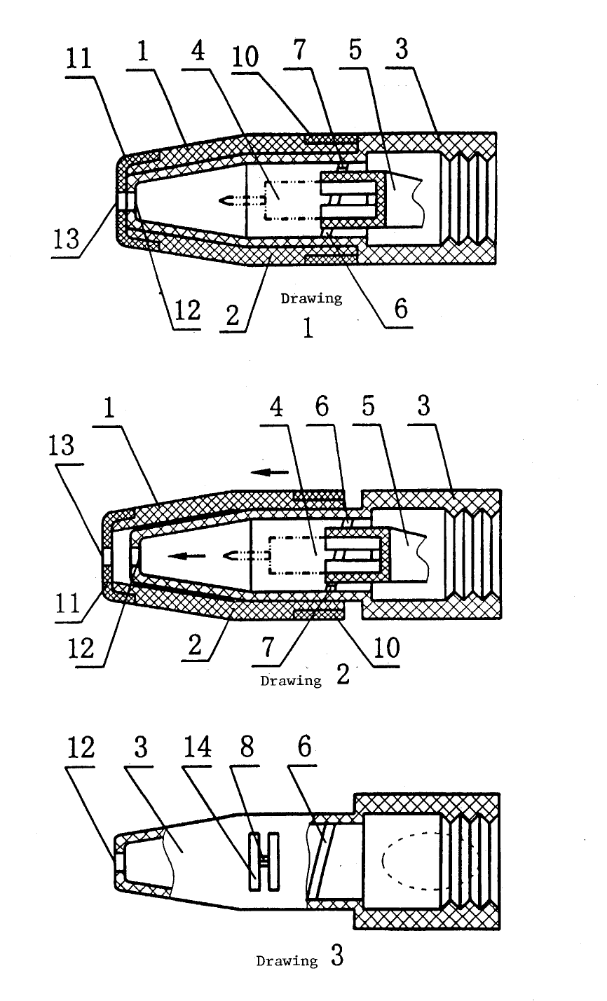

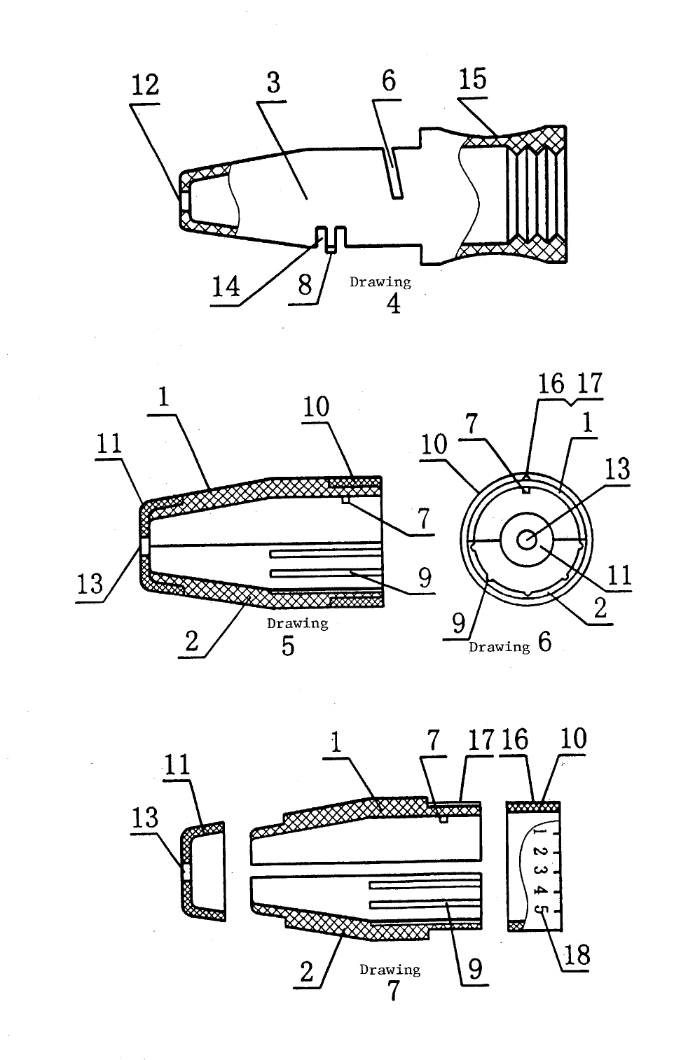

### FIELD OF THE INVENTION

This practical and new product is a reusable lancing device which uses disposable blood lancets. The lancet device is a depth adjustable structure on the head of the lancing device. The puncture depth is adjustable with this adjustable cap so that it can meet different requirements of different ages and skins.

### BACKGROUND OF THE INVENTION

The adjustable cap is a kind of puncture depth adjustable structure of lancing device. With this structure, the blood sampling depth can be well controlled and meanwhile to different people, such as adult or infant, depth can be selected to be the excellent one. The patent owner has applied for another patent “Depth adjustable cap of lancing device” on Jul. 13, 2001. The Patent Application No. is 01244815.X. This patent is mainly used to adjust puncture depth when sampling blood. So its designing idea is: add an adjustable cap with a hole B out of the hole A cap. It is the screw structure and rotary structure that join the adjustable cap and device cover. With this structure, the device can move in an interim time. Its working principle: control the puncture distance of the blood lancet with hole A. Adjustable the axes position of hole A with the screw and rotary structure so as to control the lancet's exposed length out of hole B, that is the puncture depth. To be convenient to apply, the adjustable cap is separated into parts-an adjustable set and an inner set. The hole B is positioned on the top of the adjustable set. The adjustable set can drive the inner set and lancet cover edge to move. The screw and rotary structure is set between the inner set and lancet cover. This way it is convenient to manufacture and assemble the lancing device. But such kind of inner structure brings some new problems: 1. The adjustable cap is easy to be pull off with outside force due to the tight joint between the adjustable set and inner set. 2. The adjustable veracity is not very good. 3. The whole device is not so good since it is not stable. So how to improve the inner adjustable set is the next problem that needs discussion.

### SUMMARY OF THE INVENTION

This invention is to supply a mosaic sampling depth adjustable structure which can whole join. In order to meet this requirement, the program of this practical and new product is that: an improved depth adjustable cap of lancing device, including a lancet cover with hole A at its top and adjustable cover with hole B on its top. The adjustable cover is set on the top of the lancet cover. It is the screw structure and rotary structure that join the adjustable cap and device. With this structure, the device can move in an interim time. The adjustable cover is separated into a front cover, a back ring and two pieces of semi-circle. The two pieces are joined into a cover shape. The front part is covered in one end of the cover shape. And the back ring is set in the other end. Such elements form the whole structure. The hole H is positioned on the top cover. The screw structure is set between one piece of semi-circle and lancet cover side. The rotary structure is set between the other semi-circle and the lancet cover side.

The above-mentioned technology is fit with spring lancing device with disposable blood lancet. This kind of lancing device is composed by a body, spring structure, spring and lock structure. Inside the body there is a spring chamber. The spring parts and spring perform a spring moving structure inside the spring chamber. The blood lancet is located on the lancet holder of the spring structure, so the blood lancet can be free, locked or discharge in the spring chamber. The above mentioned “lancet cover” is a component of the body head.

In order to make the head cover, back ring and two semi-circles join whole, following measures are taken:

1. The front cover is fixed on one end of the set shape which is performed by two pieces of semi-circles with a lock structure. The lock structure is a fixed structure such as lock slot, lock ring, etc.

2. To make the mosaic adjustable cover which is composed by above mentioned front cover, back ring and two semi-circles to be a good combination, ultrasonic welding can be used after assembling.

3. The above mentioned two steps can be taken at the same time.

In the above technology, a circumambient positioned structure can be set between the back ring and semi-circle. Depth marks are put on the outer edge of the back ring, so the mark position of the back ring is more exact. The mentioned circumambient positioned structure is characterized by concave and convexity. It can be performed by concave slot and convexity. The concave and convexity are set on the surface of set face of semi-circle. The other is set on the back ring.

In above program, screw structure is a relative twist of an intermittent revolving construction. The specific program is following:

1. Screw structure is made up with screw groove and convexity, one of screw groove and convexity is on inner edge of adjustable cover, and the other is on outer edge of device cap. So screw groove can be simplified forming a diagonal groove structure. Because it can simplify the manufacturing process. The screw groove and convexity have two assembling. One is that screw groove is on inner edge of adjustable cover, and convexity is on outer edger of device cap. The other is that screw groove is on outer edge of device cap, the convexity is on inner edge of adjustable cover.

2. Screw structure is made up with an inner spin and an outer spin. The inner spin is on inner edge of adjustable cover and the outer spin is on the edge of the device cap.

In the above program, a revolving position structure is a graduated construction of adjustable outer cover revolving and axial moving structure, which includes the following programs:

1. Revolving position construction is made up with groove that is set up at a distance of each other and convexity. One of the both is on inner edge of adjustable cover, the other is on outer edge of device cap. There are two assemblages of groove and convexity. One is that groove is on inner edge of adjustable cover, convexity in on outer edge of device cap. Another is that groove is on outer edge of device cap. Convexity is on inner edge of adjustable cap.

2. Revolving position construction is made up with outer edge profile of regular polygon and convexity, one is on inner edge of adjustable cover, the other is on outer edge of device cap. Outer edge profile of regular polygon means out profile and inner profile if regular polygon, when the inner profile of regular polygon is fixing in convexity, inner profile is on the inner edge of adjustable cover, and convexity is on outer edge of device cap. When the outer profile of regular polygon is fixing in convexity, the inner profile is on the outer edge of the device cap, and a convexity is on the inner edge of the adjustable cover.

This practical and new product's working principle: separate the original inner set adjustable set into four simple parts, then assemble them into one whole part and weld them with ultrasonic. With use of above-mentioned technology, this product has following advantages comparing with current technology.

1. Make original three parts-lancet cover, adjustable set and inner set to two part-lancet cover and adjustable set with ultrasonic welding. In this way, the parts join well. And the adjustable set is not easy to pull or slip off.

2. The adjustable cover is separated to front cover, back ring and two pieces of semi-circles. So the process is simpler than before. An it is easy to assemble. Facts show that the result is straight forward.

3. Separate the adjustable set into four simple parts and weld them into one whole part with ultrsonic. So the product characters and quality such as its precise and stability are improved.

### DESCRIPTION OF THE DRAWINGS

Drawing 1 is a view of adjustable cap in maximum depth;

Drawing 2 is a view of adjustable cap in minimum depth;

Drawing 3 is a view of construction of lancet cap;

Drawing 4 is a top view drawing 3;

Drawing 5 is a view of mosaic construction of adjustable cover;

Drawing 6 is a right view of drawing 5;

Drawing 7 is a separate view of adjustable cover.

In above drawings the parts are numbered as follows: (1) top semicircle; (2) bottom semi-circle; (3) lancet cover; (4) disposable blood lancet; (5) lancet holder; (6) groove; (7) convexity; (8) convex; (9) slot; (10) back ring; (11) front cover; (12) hole A; (13) hole B; (14) horizontal groover; (15) nipped position; (16) convex; (17) groove; (18) depth mark.

### DETAILED EXECUTION

The following is a further description of this product with reference to the drawings:

Sample: see drawing 1-drawing 7. An improved depth adjustable cap of lancing device, composed by a lancet cover 3 with hole A 12 on its top and an adjustable cover. The adjustable cover is set on the top of lancet cover 3. An on the top of it there is another hole B 13. The mentioned adjustable cover is separated in a front cover 11, a back ring 10, a piece of top semi-circle 1 and a piece of bottom semi-circle 2. Two semi-circles 1, 2 perform a set shape. The front cover 11 is set in one end of the set shape, and the back ring 10 is set on the other end. The hole B 13 is located in the front cover 11. There is a screw structure between the top semi-circle 1 and lancet cover 3 side. There is rotary structure between the bottom semi-circle 2 and lancet cover 3 side. The mentioned screw structure is composed by a groove 6 and a convexity 7. The skew groove 6 is located in the outer edge of lancet cover 3, and the convexity 7 is set in the inner edge of top semi-circle 1. The convexity 7 enters into the groove 6 to perform the screw function. The mentioned rotary structure is composed by a convex 8 and a slot 9. The convex 8 is in the outer edge of lancet cover 3. There are two horizontal grooves 14 on both side of the convex 8. Slot 9 is in the inner edge of bottom semicircle 2 and locates in a distance. The convex 8 and slot 9 perform a screw function. In order to emphasize the depth mark 18 on the back ring 10 it is positioned in a circumambient positioning structure set between the back ring 10 and the top semi-circle 1. This structure is composed by the groove 17 and convex 16. Groove 17 is located in the inner edge of back ring 10. Convex 16 is located in the outer edge of top semi-circle 1. When assembling, fit the above mentioned four parts of adjustable cap to be a whole one at the head of lancet cover 3. Ultrasonic welding is needed after assembling. Those in a nipped position 15 at the outer edge of lancet cover 3 back. And corners exit on the outer edge of top semi-circle 1 and bottom semi-circle 2. When adjusting the adjustable cover, the rotary position structure can make the adjustable cover move intermittently. Together the moving, “clicks” can be heard. The screw structure makes the hole B move along with the axes and control the lancet expose length.

What is claimed is:

1. An adjustable end cap for a lancing device formed of two semicircles 1, 2 and a cover 3 banded together by an adjustable depth gage back ring 10 inserted in a coaxial cover 3 having means for adjusting the depth of the movement of the lancet for multiple usage comprising:
means separating the adjustable cover from the main cover;
said back ring proportioned to coaxially engage the semicircles 1, 2;
the front cover being secured by the shape of the back ring;
a hole 13 in the end cover 11 for the semi-circle 1, 2;
said means for adjustment including a coacting internal back ring 10 engaging a cam groove 6 in communication with a spline formed of a key 16 and opposed the groove 17.

2. In the device according to claim 1,
the front cover 11 securing the top and bottom 1, 2 by grippingly receiving one end of the top and bottom 1, 2.

3. In the device according to claim 1,
a plurality of depth indicia on the back ring 10.

4. In the device according to claim 3,
a groove and rib constraining the disposable lancet to move longitudinally in its holder.

5. In the device according to claim 4,
one spline member located in the semi-circle and the other in the back ring.
